(19) United States
(12) Patent Application Publication
(10) Pub. No.: US 2023/0149655 A1
(43) Pub. Date: May 18, 2023

(54) METHODS AND APPARATUS FOR PROVIDING CONCENTRATED THERAPY GAS FOR A RESPIRATORY DISORDER

(71) Applicant: ResMed Asia Pte. Ltd., Singapore (SG)

(72) Inventors: Stephen YUEN, Singapore (SG); Rex Dael NAVARRO, Singapore (SG); Henry ROHLICH, Singapore (SG); Gordon Joseph MALOUF, Singapore (SG); Teck Wei TAN, Singapore (SG); Meiyi JIANG, Tokyo (JP); Tai Liang TING, Singapore (SG)

(73) Assignee: ResMed Asia Pte. Ltd., Singapore (SG)

(21) Appl. No.: 17/914,984

(22) PCT Filed: Apr. 5, 2021
(86) PCT No.: PCT/SG2021/050188

§ 371 Date: Sep. 27, 2022

(30) Foreign Application Priority Data
Apr. 8, 2020 (AU) 2020901121

(51) Int. Cl. A61M 16/10

(52) U.S. Cl. CPC A61M 16/101; A61M 16/107; B01D 2256/12; B01D 2259/45

(57) ABSTRACT

Oxygen concentrator apparatus provides variation in therapy gas during a breathing cycle such as by varying flow rate and/or oxygen purity of enriched air. The apparatus may include a compressor and a valve set that operates sieve bed(s) for the enriching air and to vent exhaust gas from the bed(s). The therapy gas may include released enriched air and exhaust gas. The apparatus has a supply valve to selectively release enriched air from an accumulator via a primary path to a delivery conduit. The apparatus may include a secondary path, such as with a valve, to release a portion of exhaust gas to the delivery conduit. A controller actuates the valve set to produce the enriched air, and the supply valve to release enriched air to the delivery conduit. The controller may actuate the secondary valve in anti-sync with the supply valve to release exhaust gas to the delivery conduit.

### CROSS REFERENCE TO RELATED APPLICATIONS

The present disclosure claims priority from Australia Provisional Patent Application Serial No. 2020901121, filed on 8 Apr. 2020, the entire disclosure of which is hereby incorporated herein by reference.

### FIELD OF THE TECHNOLOGY

The present technology relates generally to methods and apparatus for treating respiratory disorders, such as those involving gas adsorption or controlled pressure swing adsorption. Such methodologies may be implemented in an oxygen concentrator. In some examples, the technology more specifically concerns such methods and apparatus for generating an oxygen therapy from a portable oxygen concentrator with multiple flow paths for implementing a hybrid mode where a flow of therapy gas has characteristic(s) (e.g., purity and/or flow rate) that may differ during inspiration (or part of inspiration) relative to non-inspiration times or expiration.

### DESCRIPTION OF THE RELATED ART

### Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive. Rapidly progressive disorders are characterised by muscle impairment that worsens over months and results in death within a few years (e.g. Amyotrophic lateral sclerosis (ALS) and Duchenne muscular dystrophy (DMD) in teenagers). Variable or slowly progressive disorders are characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Respiratory Therapies

In respiratory therapies known as “flow” therapies, the interface to the patient's airways is ‘open’ (unsealed) and the respiratory therapy may supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate.

Another form of flow therapy is long-term oxygen therapy (LTOT) or supplemental oxygen therapy. Doctors may prescribe a continuous flow of oxygen enriched air at a specified oxygen purity (from 21%, the oxygen fraction in ambient air, to 100%) at a specified flow rate (e.g., 1 litre per minute (LPM), 2 LPM, 3 LPM, etc.) to be delivered to the patient's airway.

### Respiratory Therapy Systems

Respiratory flow therapies may be provided by a respiratory therapy system or device. A respiratory therapy system as described herein may comprise an oxygen source, an air circuit, and a patient interface.

### Air Circuit

An air circuit is a conduit or a tube constructed and arranged to allow, in use, a flow of conditioned or enriched air to travel between two components of a respiratory therapy system such as the oxygen source and the patient interface.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. For flow therapies such as nasal HFT or LTOT, the patient interface is configured to insufflate the nares but specifically to avoid a complete seal. One example of such a patient interface is a nasal cannula.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may serve as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the sieve beds. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a canister containing a gas separation adsorbent that attracts nitrogen more strongly than it does oxygen, part or all of the nitrogen will stay in the canister, and the gas coming out of the canister will be enriched in oxygen. When the sieve bed reaches the end of its capacity to adsorb nitrogen, the adsorbed nitrogen may be desorbed by venting. The sieve bed is then ready for another cycle of producing oxygen enriched air. By alternating pressurization cycles of the canisters in a two-canister system, one canister can be separating oxygen while the other canister is being vented (resulting in a near-continuous separation of oxygen from the air). In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for depressurizing the sieve beds.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and provide mobility for patients (users) during use. In order to make these devices small for mobility, the various systems necessary for the production of oxygen enriched air are condensed. POCs seek to utilize their produced oxygen as efficiently as possible, in order to minimise weight, size, and power consumption. In some implementations, this may be achieved by delivering the oxygen as series of pulses, each pulse or “bolus” timed to coincide with the onset of inhalation. This therapy mode is known as pulsed oxygen delivery (POD) or demand mode.

Continuous flow mode long-term oxygen therapy is advantageous for clinical reasons, e.g. reassuring patients they are receiving therapy, and relieving anxiety. However, continuous flow mode is draining of battery life and thus is more suitable for stationary devices. A need therefore exists for a portable oxygen concentrator capable of emulating the benefits of continuous flow mode with reasonable battery life.

### SUMMARY OF THE TECHNOLOGY

Examples of the present technology may provide methods and apparatus for controlled operations of an oxygen concentrator, such as a portable oxygen concentrator. In particular, the technology provides methods and apparatus for a portable oxygen concentrator configured to deliver long-term oxygen therapy in a mode of delivery referred to herein as hybrid mode, while maintaining acceptable battery life. Hybrid mode therapy is a breath-synchronised therapy in which there is a non-zero inter-bolus flow of gas to the patient as well as boluses delivered in synchrony with inhalation as in POD mode. Hybrid mode therapy may be delivered according to a bilevel purity species, a bilevel flow rate species, or species intermediate between those two species.

All species of hybrid mode therapy present a challenge to traditional methods of detecting the onset of user inhalation. Examples of the present technology may therefore also include a sensor configuration that allows accurate detection of the onset of inhalation in the various sub-modes of hybrid mode therapy.

Some implementations of the present technology may include an oxygen concentrator for providing a therapy gas to a delivery conduit for patient inhalation. The oxygen concentrator may include a compressor configured to generate a pressurised air stream. The oxygen concentrator may include one or more sieve beds. The one or more sieve beds may include adsorbent material configured to preferentially adsorb a component gas from the pressurised air stream, thereby producing oxygen enriched air from the pressurised air stream. The oxygen concentrator may include a valve set. The valve set may be configured to selectively pneumatically couple the compressor to the one or more sieve beds so as to selectively convey the pressurised air stream to the one or more sieve beds. The valve set may be configured to selectively vent exhaust gas to atmosphere from an exhaust outlet of the one or more sieve beds. The oxygen concentrator may include an accumulator pneumatically coupled to the one or more sieve beds so as to receive the oxygen enriched air produced from a product outlet of the one or more sieve beds. The oxygen concentrator may include a supply valve configured to selectively release oxygen enriched air from the accumulator via a primary flow path and then to the delivery conduit. The oxygen concentrator may include a secondary flow path configured to pass a portion of the exhaust gas from the exhaust outlet to the delivery conduit. The oxygen concentrator may include a controller operably coupled to the valve set and the supply valve. The controller may be configured to selectively actuate the valve set in a periodic pattern so as to produce oxygen enriched air for receiving by the accumulator and vent exhaust gas from the one or more sieve beds. The controller may be configured to selectively actuate the supply valve to release oxygen enriched air from the accumulator to the delivery conduit in synchrony with inhalation of the patient. The therapy gas may include the released oxygen enriched air and the portion of the exhaust gas.

In some implementations, the therapy gas may be provided to the delivery conduit in a hybrid mode wherein the therapy gas flows to the delivery conduit at least during patient inspiration and patient expiration. The hybrid mode may vary a characteristic of the therapy gas. The varied characteristic may be oxygen purity. The varied oxygen purity may include a first oxygen purity during at least a portion of patient inspiration and a second oxygen purity after the portion of patient inspiration. The first oxygen purity may be a purity in a range of about 50 percent to about 99 percent. The second oxygen purity may be a purity in a range of about 4 percent to 35 percent. The primary flow path may be configured to provide the therapy gas with the first oxygen purity. The secondary flow path may be configured to provide the therapy gas with the second oxygen purity. The secondary flow path may include a secondary valve configured to selectively release the portion of the exhaust gas to the delivery conduit. The controller may be further configured to selectively actuate the secondary valve in anti-sync with actuation of the supply valve to release the portion of the exhaust gas to the delivery conduit. The supply valve and the secondary valve may be implemented as a three-way valve configured to release either the oxygen enriched air or the portion of the exhaust gas to the delivery conduit.

In some implementations, the oxygen concentrator may further include a pressure sensor configured to generate a signal representative of a difference in pressure between a sense port and a reference port thereof, the sense port being connected to the delivery conduit, and the reference port being coupled to a flow path of the oxygen concentrator that may be downstream of the supply valve. The controller may be further configured to detect onset of inhalation from the generated pressure difference signal and to actuate the supply valve based on the detected onset of inhalation. The controller may be configured to detect onset of inhalation by detecting a drop in the generated pressure difference signal. The reference port of the pressure sensor may be connected to a downstream side of the supply valve via a flow restrictor. The controller may be configured to actuate the secondary valve in anti-sync with actuation of the supply valve in response to user activation of a control on an interface of the oxygen concentrator.

In some implementations, the oxygen concentrator may further include a flow restrictor within the secondary flow path and in line with the secondary valve. The flow restrictor may be configured such that a flow rate of exhaust gas when released to the delivery conduit may be approximately equal to a flow rate of the oxygen enriched air when released to the delivery conduit. The oxygen concentrator may include a further secondary valve configured to selectively release oxygen enriched air from the accumulator to the delivery conduit via a flow restrictor. The controller may be further configured to selectively actuate the further secondary valve in anti-sync with actuation of the supply valve to release oxygen enriched air to the delivery conduit. The hybrid mode may vary a further characteristic of the therapy gas. The varied further characteristic may be flow rate of the therapy gas.

Some implementations of the present technology may include apparatus for providing a therapy gas. The apparatus may include means for generating a pressurised air stream such as a motor operated compressor as described in more detail herein. The apparatus may include means for preferentially adsorbing a component gas from the pressurised air stream, thereby producing oxygen enriched air from the pressurised air stream such as one or more sieve beds as described in more detail herein. The apparatus may include means for selectively pneumatically coupling, in a periodic pattern, the means for preferentially adsorbing with (a) the means for generating so as to selectively convey the pressurised air stream to the means for preferentially adsorbing, and (b) an exhaust outlet to atmosphere for selectively venting exhaust gas to atmosphere from the means for preferentially adsorbing, so as to produce oxygen enriched air within the means for preferentially adsorbing, such as a controller and a set of valves described in more detail herein. The apparatus may include means for accumulating the oxygen enriched air, such as an accumulator as described in more detail herein, produced from a product outlet of the means for preferentially adsorbing. The apparatus may include means for selectively releasing oxygen enriched air from the means for accumulating to a delivery conduit for a patient in synchrony with inhalation of the patient, such as a supply valve and a controller described in more detail herein. The apparatus may include means for passing a portion of the exhaust gas to the delivery conduit, such as a secondary flow path as described in more detail herein. The therapy gas may include the released oxygen enriched air from the means for accumulating and the portion of the exhaust gas.

Some implementations of the present technology may include an oxygen concentrator for producing a therapy gas for a patient. The oxygen concentrator may include a compressor configured to generate a pressurised air stream. The oxygen concentrator may include one or more sieve beds. The one or more sieve beds may include adsorbent material configured to preferentially adsorb a component gas from the pressurised air stream, thereby producing oxygen enriched air from the pressurised air stream. The oxygen concentrator may include a valve set configured to selectively pneumatically couple the compressor to the one or more sieve beds so as to selectively convey the pressurised air stream to the one or more sieve beds. The oxygen concentrator may include an accumulator pneumatically coupled to the one or more sieve beds so as to receive the oxygen enriched air produced by one or more sieve beds. The oxygen concentrator may include a supply valve configured to selectively release oxygen enriched air from the accumulator, via a primary path, to a delivery conduit for the patient. The oxygen concentrator may include a secondary valve configured to selectively release oxygen enriched air from the accumulator, via a secondary path, to the delivery conduit for the patient. The oxygen concentrator may include a controller operably coupled to the valve, the supply valve, and the secondary valve. The controller may be configured to selectively actuate the valve set in a periodic pattern so as to produce oxygen enriched air in the accumulator. The controller may be configured to selectively actuate the supply valve to release oxygen enriched air to the delivery conduit in synchrony with inhalation of the patient. The controller may be configured to selectively actuate the secondary valve in anti-sync with actuation of the supply valve to release oxygen enriched air to the delivery conduit.

In some implementations, the therapy gas may be provided to the delivery conduit in a hybrid mode wherein the therapy gas flows to the delivery conduit at least during patient inspiration and patient expiration; and wherein the hybrid mode varies a characteristic of the therapy gas. The varied characteristic may be a flow rate of the therapy gas. The flow characteristic of the primary path may be different from a flow characteristic of the secondary path. The oxygen concentrator may further include a flow restrictor within the secondary path and in line with the secondary valve. The flow restrictor may be configured such that a flow rate of oxygen enriched air when released to the delivery conduit via the secondary valve may be substantially lower than a flow rate of the oxygen enriched air when released to the delivery conduit via the supply valve. The supply valve and the secondary valve may be implemented as a three-way valve configured to release oxygen enriched air to the delivery conduit.

In some implementations, the oxygen concentrator may further include a pressure sensor configured to generate a signal representative of a difference in pressure between a sense port and a reference port thereof. The sense port may be connected to the delivery conduit and the reference port may be coupled to a flow path of the oxygen concentrator that may be downstream of the supply valve. The controller may be further configured to detect onset of inhalation from the generated pressure difference signal and to actuate the supply valve based on the detected onset of inhalation. The controller may be configured to detect onset of inhalation by detecting a drop in the generated pressure difference signal. The reference port of the pressure sensor may be connected to a downstream side of the supply valve via a flow restrictor. The controller may be configured to actuate the secondary valve in anti-sync with actuation of the supply valve in response to user activation of a control on an interface of the oxygen concentrator. The oxygen concentrator may further include a further secondary valve configured to selectively release a portion of exhaust gas from the one or more sieve beds to the delivery conduit, wherein the controller may be further configured to selectively actuate the further secondary valve in anti-sync with actuation of the supply valve to release the portion of the exhaust gas to the delivery conduit. The hybrid mode may vary a further characteristic of the therapy gas. The varied further characteristic may be oxygen purity of the therapy gas.

Some implementations of the present technology may include apparatus. The apparatus may include means for generating a pressurised air stream. The apparatus may include means for preferentially adsorbing a component gas from the pressurised air stream, thereby producing oxygen enriched air from the pressurised air stream. The apparatus may include means for selectively pneumatically coupling, in a periodic pattern, the means for preferentially adsorbing with the means for generating to selectively convey the pressurised air stream to the means for preferentially adsorbing so as to produce oxygen enriched air in the means for preferentially absorbing. The apparatus may include means for accumulating the oxygen enriched air produced by the means for preferentially adsorbing. The apparatus may include primary means for selectively releasing, in synchrony with inhalation of a patient, oxygen enriched air from the means for accumulating to a delivery conduit for the patient. The apparatus may include secondary means for selectively releasing, in anti-sync with actuation of the primary means for selectively releasing, oxygen enriched air from the means for accumulating to the delivery conduit for the patient.

Some implementations of the present technology may include an oxygen concentrator. The oxygen concentrator may include a compressor configured to generate a pressurised air stream. The apparatus may include one or more sieve beds. The one or more sieve beds may include adsorbent material configured to preferentially adsorb a component gas from the pressurised air stream, thereby producing oxygen enriched air from the pressurised air stream. The apparatus may include a valve set configured to selectively pneumatically couple the compressor to the one or more sieve beds so as to selectively convey the pressurised air stream to the one or more sieve beds. The apparatus may include an accumulator pneumatically coupled to the one or more sieve beds so as to receive the oxygen enriched air produced by the one or more sieve beds. The apparatus may include a supply valve configured to selectively release oxygen enriched air from the accumulator to a delivery conduit for a patient. The apparatus may include a secondary path configured to convey a flow of gas to the delivery conduit for the patient. The apparatus may include a pressure sensor configured to generate a signal representative of a difference in pressure between a sense port and a reference port thereof. The sense port may be connected to the delivery conduit. The reference port may be coupled to a flow path of the oxygen concentrator that may be downstream of the supply valve. The apparatus may include a controller operably coupled to the valve set and the supply valve. The controller may be configured to selectively actuate the valve set in a periodic pattern so as to produce oxygen enriched air for the accumulator. The controller may be configured to detect onset of inhalation of the patient from the generated pressure difference signal. The controller may be configured to selectively actuate the supply valve to release oxygen enriched air to the delivery conduit in synchrony with inhalation of the patient.

In some implementations, the controller may be further configured to actuate the supply valve based on the detected onset of inhalation. The controller may be configured to detect onset of inhalation by detecting a drop in the generated pressure difference signal. The reference port of the pressure sensor may be connected to a downstream side of the supply valve via a flow restrictor. The secondary path may include a secondary valve configured to selectively release exhaust gas from the one or more sieve beds to the delivery conduit. The secondary path may further include a further secondary valve configured to selectively release oxygen enriched air from the accumulator to the delivery conduit via a flow restrictor. The secondary path may include a secondary valve configured to selectively release oxygen enriched air from the accumulator to the delivery conduit via a flow restrictor.

Some implementations of the present technology may include apparatus. The apparatus may include means for generating a pressurised air stream. The apparatus may include means for preferentially adsorbing a component gas from the pressurised air stream, thereby producing oxygen enriched air from the pressurised air stream. The apparatus may include means for selectively pneumatically coupling, in a periodic pattern, the means for preferentially adsorbing with the means for generating so as to selectively convey the pressurised air stream to the means for preferentially adsorbing so as to produce oxygen enriched air in the means for preferentially absorbing. The apparatus may include means for accumulating the oxygen enriched air produced by the means for preferentially adsorbing. The apparatus may include means for selectively releasing oxygen enriched air from the means for accumulating to a delivery conduit for a patient. The apparatus may include secondary means for conveying a flow of gas to the delivery conduit for the patient. The apparatus may include means for generating a signal representative of a difference in pressure between a sense port and a reference port thereof. The sense port may be connected to the delivery conduit. The apparatus may include means for detecting onset of inhalation of the patient from the generated pressure difference signal and for selectively actuating the means for selectively releasing oxygen enriched air to release oxygen enriched air to the delivery conduit in synchrony with inhalation of the patient.

Another general aspect includes an oxygen concentrator. The oxygen concentrator includes a compressor configured to generate a pressurised air stream. The oxygen concentrator also includes at least one sieve bed, the or each each sieve bed including adsorbent material configured to preferentially adsorb a component gas from the pressurised air stream, thereby producing oxygen enriched air from the pressurised air stream. The oxygen concentrator also includes a valve configured to: selectively pneumatically couple the compressor to the or each sieve bed so as to selectively convey the pressurised air stream to the sieve bed, and selectively vent exhaust gas from the or each sieve bed. The oxygen concentrator also includes an accumulator pneumatically coupled to the or each sieve bed so as to receive the oxygen enriched air produced by the or each sieve bed. The oxygen concentrator also includes a supply valve configured to selectively release oxygen enriched air from the accumulator to a patient via a delivery conduit. The oxygen concentrator also includes a secondary valve configured to selectively release a portion of the exhaust gas to the delivery conduit. The oxygen concentrator also includes a controller operably coupled to the valve, the supply valve, and the secondary valve, the controller configured to: selectively actuate the valve in a periodic pattern so as to produce oxygen enriched air in the accumulator, selectively actuate the supply valve to release oxygen enriched air to the delivery conduit in synchrony with inhalation of the patient, and selectively actuate the secondary valve in anti-sync with actuation of the supply valve to release the portion of the exhaust gas to the delivery conduit.

One general aspect includes apparatus. The apparatus also includes means for generating a pressurised air stream. The apparatus also includes means for preferentially adsorbing a component gas from the pressurised air stream, thereby producing oxygen enriched air from the pressurised air stream. The apparatus also includes means for selectively pneumatically coupling the means for generating to the means for preferentially adsorbing so as to selectively convey the pressurised air stream to the means for preferentially adsorbing. The apparatus also includes means for selectively venting exhaust gas from the means for preferentially adsorbing. The apparatus also includes means for receiving the oxygen enriched air produced by the means for preferentially adsorbing. The apparatus also includes means for selectively releasing oxygen enriched air from the means for receiving to a patient via a delivery conduit. The apparatus also includes means for selectively releasing a portion of the exhaust gas to the delivery conduit. The apparatus also includes means for selectively actuating the means for selectively pneumatically coupling in a periodic pattern so as to produce oxygen enriched air in the means for receiving. The apparatus also includes means for selectively actuating the means for selectively releasing oxygen enriched air to release oxygen enriched air to the delivery conduit in synchrony with inhalation of the patient. The apparatus also includes means for selectively actuating the means for selectively releasing exhaust gas in anti-sync with actuation of the means for selectively releasing oxygen enriched air to release the portion of the exhaust gas to the delivery conduit.

One general aspect includes an oxygen concentrator. The oxygen concentrator also includes a compressor configured to generate a pressurised air stream. The oxygen concentrator also includes at least one sieve bed, the or each each sieve bed including adsorbent material configured to preferentially adsorb a component gas from the pressurised air stream, thereby producing oxygen enriched air from the pressurised air stream. The oxygen concentrator also includes a valve configured to selectively pneumatically couple the compressor to the or each sieve bed so as to selectively convey the pressurised air stream to the sieve bed. The oxygen concentrator also includes an accumulator pneumatically coupled to the or each sieve bed so as to receive the oxygen enriched air produced by the or each sieve bed. The oxygen concentrator also includes a supply valve configured to selectively release oxygen enriched air from the accumulator to a patient via a delivery conduit. The oxygen concentrator also includes a secondary valve configured to selectively release oxygen enriched air from the accumulator to the patient via the delivery conduit. The oxygen concentrator also includes a controller operably coupled to the valve, the supply valve, and the secondary valve, the controller configured to: selectively actuate the valve in a periodic pattern so as to produce oxygen enriched air in the accumulator, selectively actuate the supply valve to release oxygen enriched air to the delivery conduit in synchrony with inhalation of the patient, and selectively actuate the secondary valve in anti-sync with actuation of the supply valve to release oxygen enriched air to the delivery conduit.

One general aspect includes apparatus. The apparatus also includes means for generating a pressurised air stream. The apparatus also includes means for preferentially adsorbing a component gas from the pressurised air stream, thereby producing oxygen enriched air from the pressurised air stream. The apparatus also includes means for selectively pneumatically coupling the means for generating to the means for preferentially adsorbing so as to selectively convey the pressurised air stream to the means for preferentially adsorbing. The apparatus also includes means for receiving the oxygen enriched air produced by the means for preferentially adsorbing. The apparatus also includes means for selectively releasing oxygen enriched air from the means for receiving to a patient via a delivery conduit. The apparatus also includes secondary means for selectively releasing oxygen enriched air from the means for receiving to a patient via a delivery conduit. The apparatus also includes means for selectively actuating the means for selectively pneumatically coupling in a periodic pattern so as to produce oxygen enriched air in the means for receiving. The apparatus also includes means for selectively actuating the means for selectively releasing oxygen enriched air to release oxygen enriched air to the delivery conduit in synchrony with inhalation of the patient. The apparatus also includes means for selectively actuating the secondary means for selectively releasing oxygen enriched air in anti-sync with actuation of the means for selectively releasing oxygen enriched air to release oxygen enriched air to the delivery conduit.

One general aspect includes an oxygen concentrator. The oxygen concentrator also includes a compressor configured to generate a pressurised air stream. The oxygen concentrator also includes at least one sieve bed, the or each each sieve bed including adsorbent material configured to preferentially adsorb a component gas from the pressurised air stream, thereby producing oxygen enriched air from the pressurised air stream. The oxygen concentrator also includes a valve configured to selectively pneumatically couple the compressor to the or each sieve bed so as to selectively convey the pressurised air stream to the sieve bed. The oxygen concentrator also includes an accumulator pneumatically coupled to the or each sieve bed so as to receive the oxygen enriched air produced by the or each sieve bed. The oxygen concentrator also includes a supply valve configured to selectively release oxygen enriched air from the accumulator to a patient via a delivery conduit. The oxygen concentrator also includes a secondary path configured to convey a flow of gas to the patient via the delivery conduit. The oxygen concentrator also includes a pressure sensor configured to generate a signal representative of a difference in pressure between a sense port and a reference port thereof, the sense port being connected to the delivery conduit. The oxygen concentrator also includes a controller operably coupled to the valve and the supply valve, the controller configured to: selectively actuate the valve in a periodic pattern so as to produce oxygen enriched air in the accumulator, detect onset of inhalation of the patient from the generated pressure difference signal, and selectively actuate the supply valve to release oxygen enriched air to the delivery conduit in synchrony with inhalation of the patient.

One general aspect includes apparatus. The apparatus also includes means for generating a pressurised air stream. The apparatus also includes means for preferentially adsorbing a component gas from the pressurised air stream, thereby producing oxygen enriched air from the pressurised air stream. The apparatus also includes means for selectively pneumatically coupling the means for generating to the means for preferentially adsorbing so as to selectively convey the pressurised air stream to the means for preferentially adsorbing. The apparatus also includes means for receiving the oxygen enriched air produced by the means for preferentially adsorbing. The apparatus also includes means for selectively releasing oxygen enriched air from the means for receiving to a patient via a delivery conduit. The apparatus also includes means for conveying a flow of gas to the patient via the delivery conduit. The apparatus also includes means for generating a signal representative of a difference in pressure between a sense port and a reference port thereof, the sense port being connected to the delivery conduit. The apparatus also includes means for selectively actuating the means for selectively pneumatically coupling in a periodic pattern so as to produce oxygen enriched air in the means for receiving. The apparatus also includes means for detecting onset of inhalation of the patient from the generated pressure difference signal. The apparatus also includes means for selectively actuating the means for selectively releasing oxygen enriched air to release oxygen enriched air to the delivery conduit in synchrony with inhalation of the patient.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of implementations and upon reference to the accompanying drawings in which similar reference numerals indicate similar components:

FIG. 1A depicts an oxygen concentrator in accordance with one form of the present technology.

FIG. 1B is a schematic diagram of the components of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 1I is a perspective view of a disassembled canister system for the oxygen concentrator of FIG. 1A.

FIG. 1J is an end view of the canister system of FIG. 1I.

FIG. 1K is an assembled view of the canister system end depicted in FIG. 1J.

FIG. 1L is a view of an opposing end of the canister system of FIG. 1I to that depicted in FIGS. 1J and 8K.

FIG. 1M is an assembled view of the canister system end depicted in FIG. 1L.

FIG. 1N depicts an example control panel for the oxygen concentrator of FIG. 1A.

FIG. 1O depicts a connected POC therapy system that includes the oxygen concentrator of FIG. 1A.

FIG. 2 contains a graph illustrating the bilevel purity implementation of hybrid delivery mode according to one aspect of the present technology.

FIG. 3 is a schematic diagram of a modification to the outlet system of FIG. 1F according to one implementation of the present technology.

FIG. 4 contains a graph illustrating the bilevel flow rate implementation of hybrid delivery mode according to one aspect of the present technology.

FIG. 5 is a schematic diagram of a modification to the outlet system of FIG. 1F according to one implementation of the present technology.

FIG. 6 contains a graph illustrating various modes of delivery of oxygen enriched air by an oxygen concentrator.

FIG. 7 is a schematic diagram of a modification to the outlet system of FIG. 1F according to one implementation of a combination of the outlet systems of FIGS. 3 and 5 of the present technology.

### DETAILED DESCRIPTION OF THE IMPLEMENTATIONS

Examples implementations of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

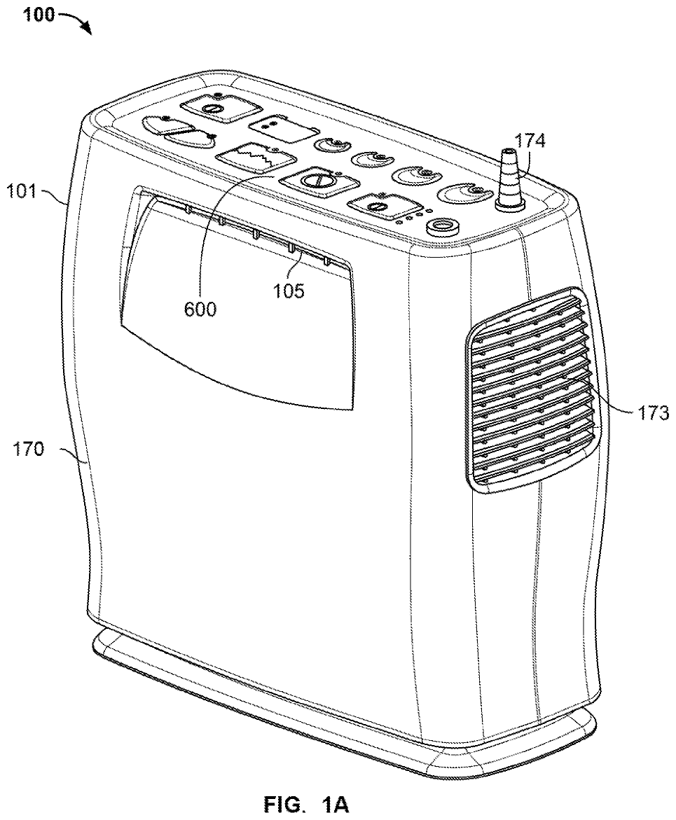

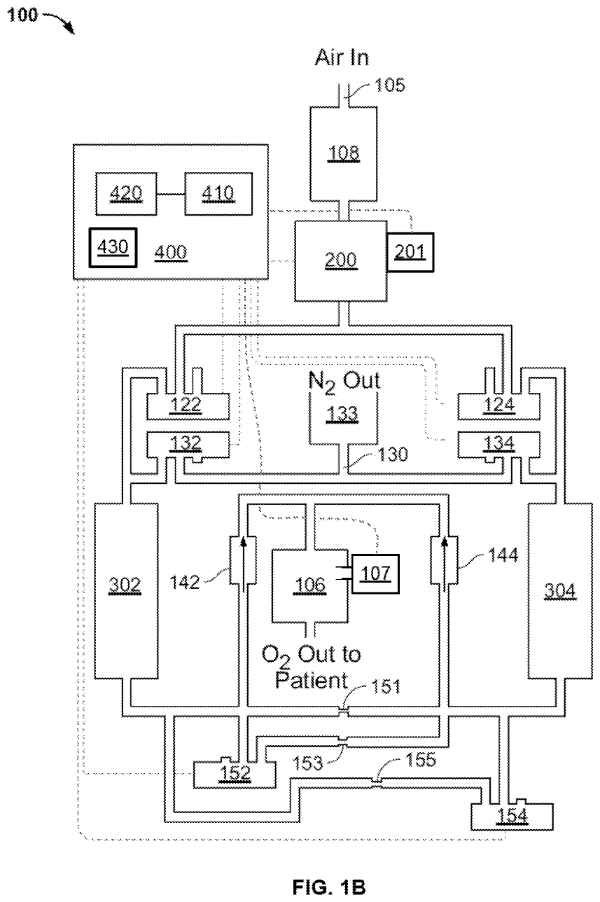

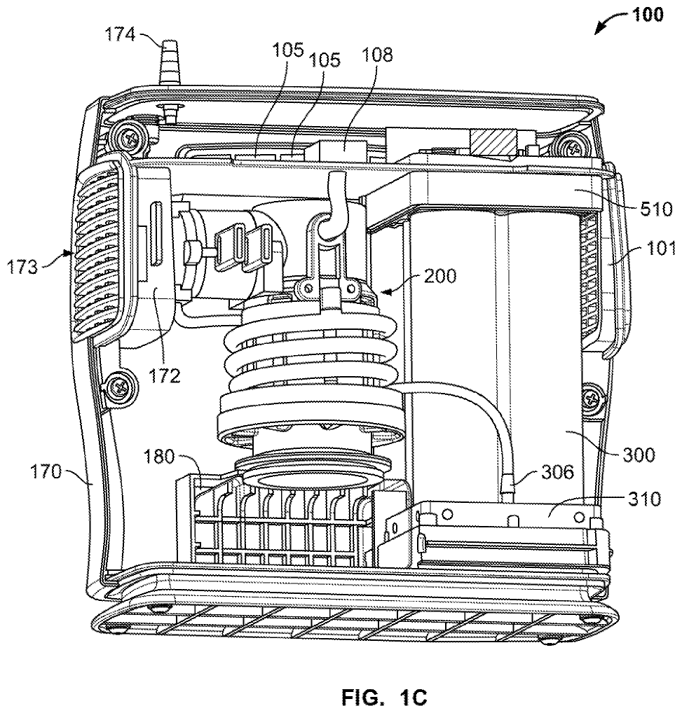

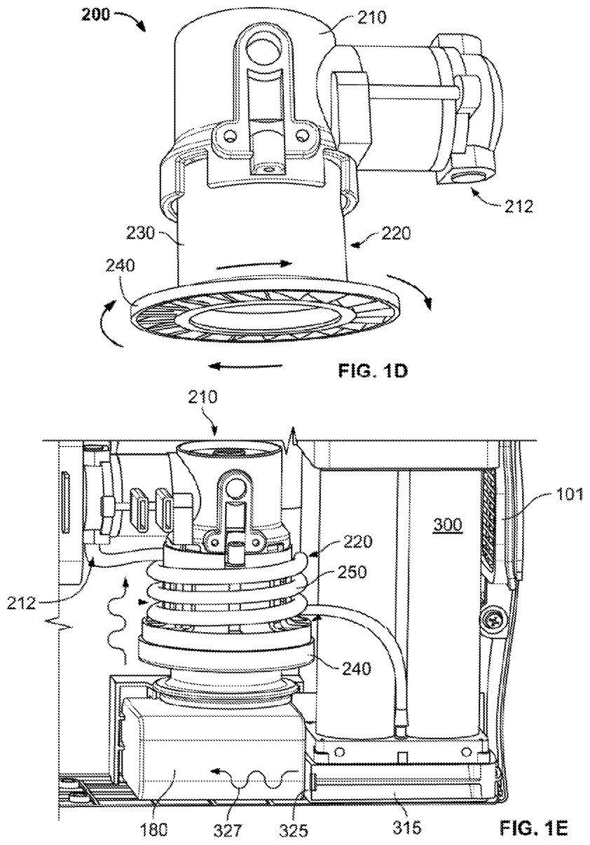

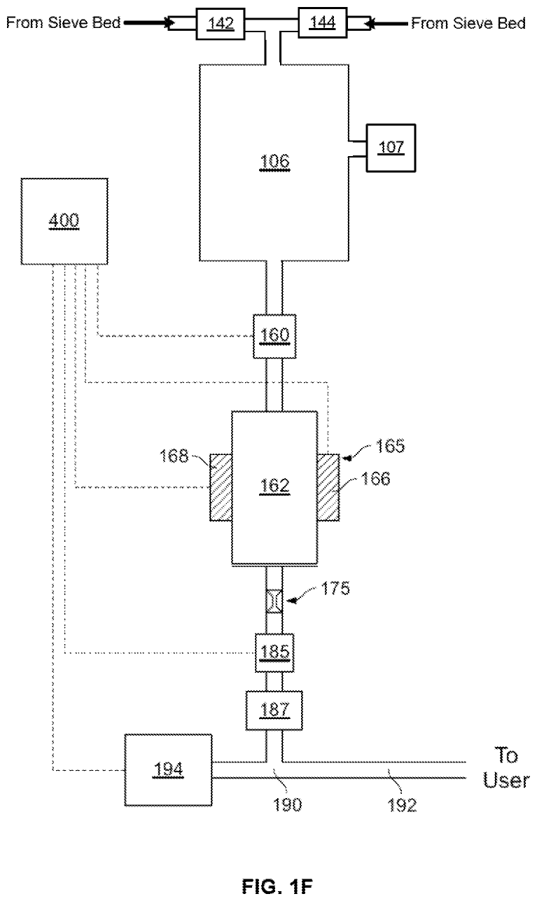

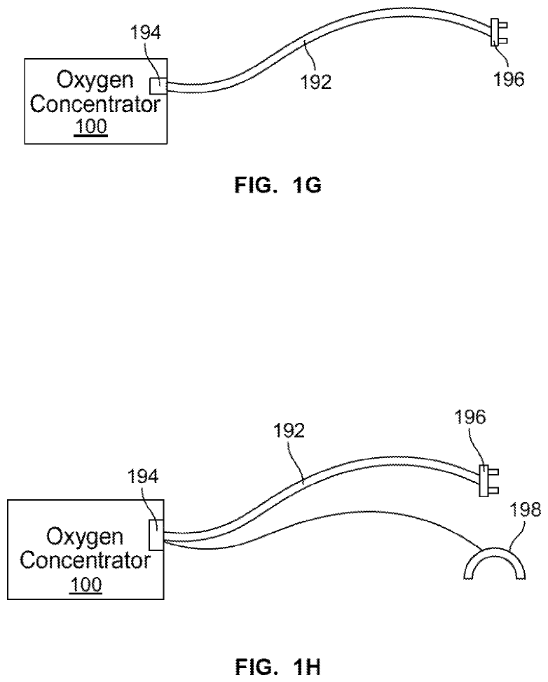

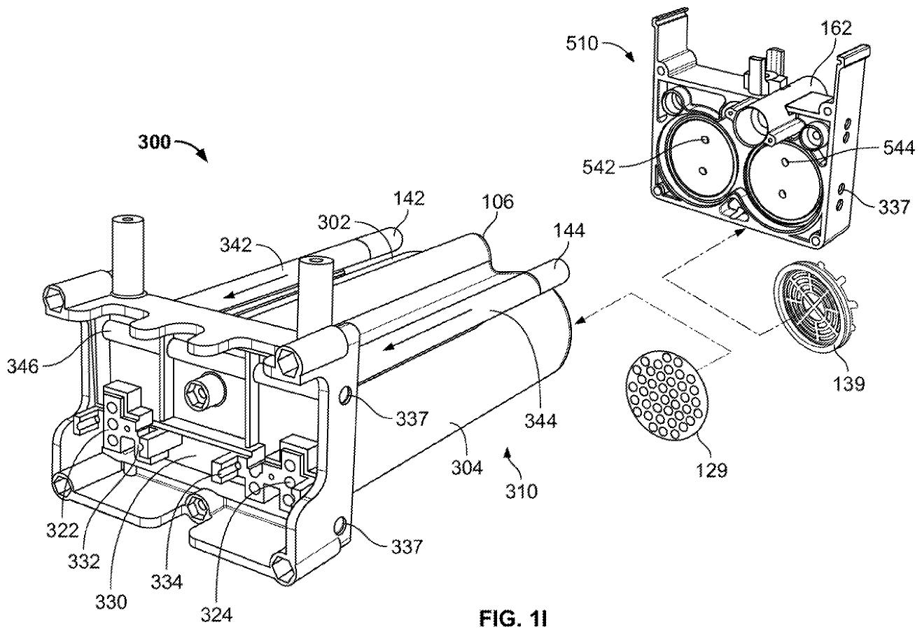

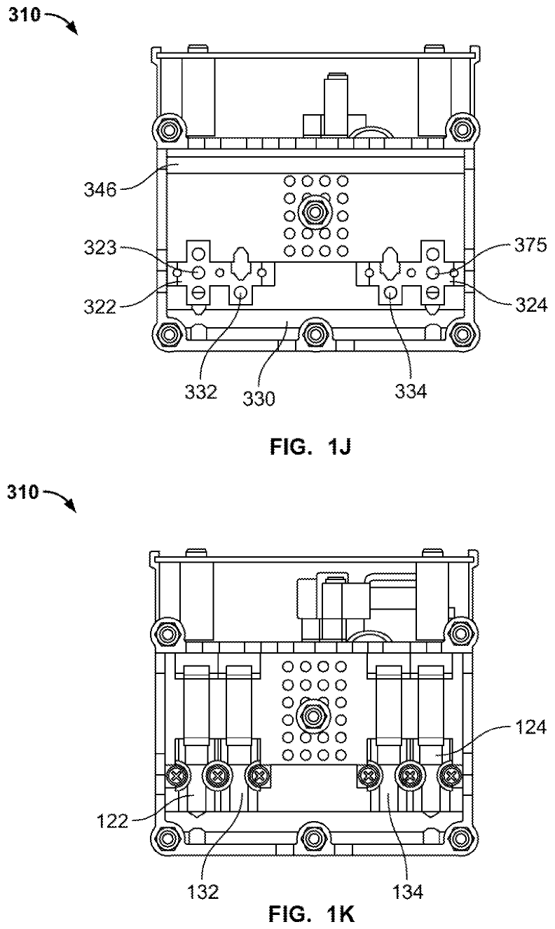

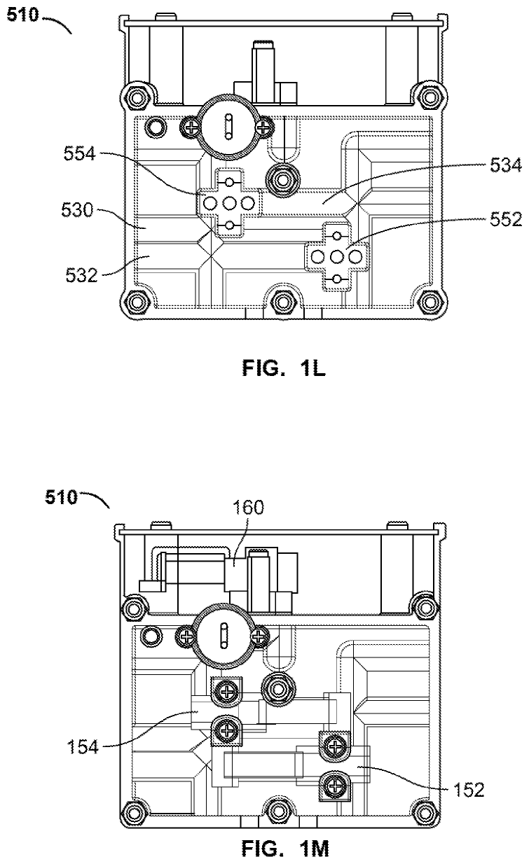

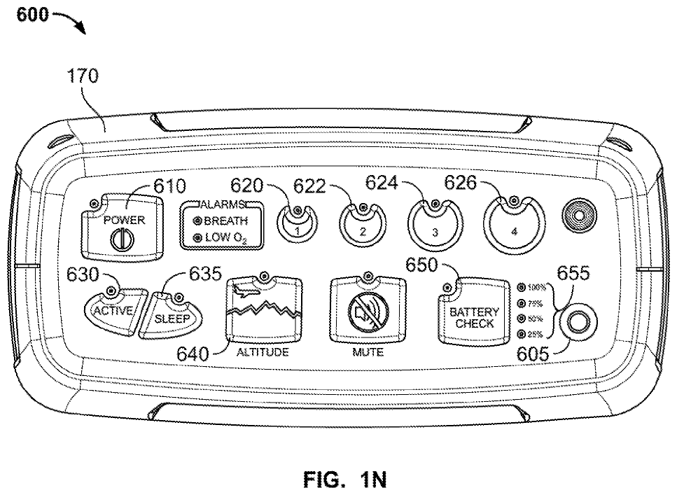

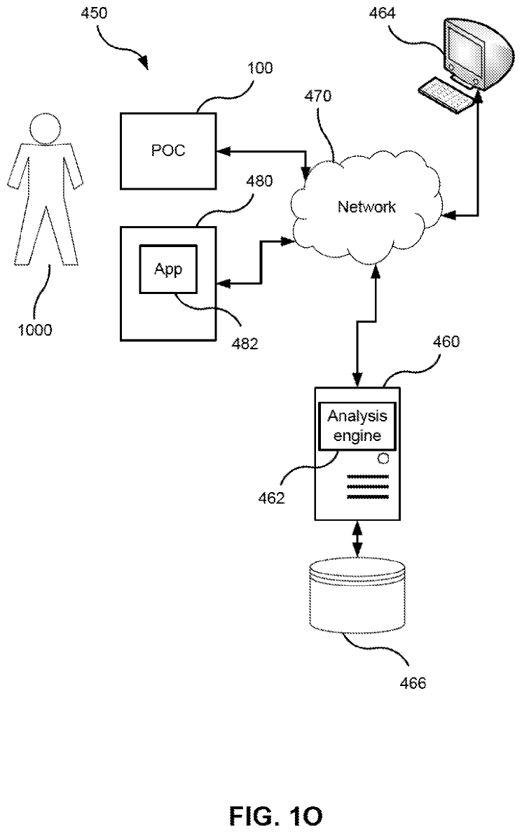

FIGS. 1A-8N illustrate an implementation of an oxygen concentrator 100. As described herein, oxygen concentrator 100 uses pressure swing adsorption (PSA) processes to produce oxygen enriched air. However, in other embodiments, oxygen concentrator 100 may be modified such that it uses vacuum swing adsorption (VSA) processes or vacuum pressure swing adsorption (VPSA) processes to produce oxygen enriched air.

### Outer Housing

FIG. 1A depicts an implementation of an outer housing 170 of an oxygen concentrator 100. In some implementations, outer housing 170 may be comprised of a light-weight plastic. Outer housing 170 includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Components

FIG. 1B illustrates a schematic diagram of components of an oxygen concentrator 100, according to an implementation. Oxygen concentrator 100 may concentrate oxygen within an air stream to provide oxygen enriched air to a user.

Oxygen concentrator 100 may be a portable oxygen concentrator. For example, oxygen concentrator 100 may have a weight and size that allows the oxygen concentrator to be carried by hand and/or in a carrying case during use. As discussed further herein, such a device typically operates with an included power supply that provides power to the oxygen concentrator using one or more batteries, such as Lithium ion batteries, which are typically rechargeable. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

Oxygen enriched air may be produced from ambient air by pressurising ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a water adsorbent material (such as a polymer water adsorbent material or a zeolite material) may be used to both adsorb water from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch gauge pressure (psig). Other pressures may also be used, depending on the type of gas separation adsorbent disposed in the canisters.

The oxygen concentrator may typically include a valve set of one more valves for directing the pressurized air for the processes of the oxygen concentrator so as to produce the oxygen enriched air. For example, coupled to each canister 302/304 are inlet valves 122/124 and outlet valves 132/134. As shown in FIG. 1B, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be generated to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400. The controller 400 may include a transceiver 430 that may communicate with external devices to transmit data collected by the processor 410 or receive instructions from an external device for the processor 410.

Check valves 142 and 144 are coupled to canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere via the canister's exhaust outlet while canister 302 is being pressurized. Canister 302 is pressurized until the pressure in canister is sufficient to open check valve 142. Oxygen enriched air produced in canister 302 exits from the canister's product outlet and passes through a check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to desorb nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302 from its exhaust outlet, outlet valve 132 may be opened allowing exhaust gas to exit the canister to atmosphere through concentrator outlet 130. In an implementation, the vented exhaust gas may be directed through muffler 133 to reduce the noise produced by releasing the pressurized gas from the canister. As gas is released from canister 302, the pressure in the canister 302 drops, allowing nitrogen to become desorbed from the gas separation adsorbent. The vented exhaust gas exits the oxygen concentrator through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, nitrogen removal may be assisted using an oxygen enriched air stream that is introduced into the canister from the other canister or stored oxygen enriched air.

In an exemplary implementation, a portion of the oxygen enriched air may be transferred from canister 302 to canister 304 when canister 304 is being vented of exhaust gas. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalising the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. Oxygen concentrator 100 includes a compression system 200, a canister system 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

### Compression System

In some implementations, compression system 200 includes one or more compressors. In another implementation, compression system 200 includes a single compressor, coupled to all of the canisters of canister system 300. Turning to FIGS. 1D and 8E, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 1B, which sends operating signals to the motor to control the operation of the motor. For example, controller 400 may send signals to motor 220 to: turn the motor on, turn motor the off, and set the operating speed of motor. Thus, as illustrated in FIG. 1B, the compression system may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system via the motor 220 based on the speed signal and/or any other sensor signal of the oxygen concentrator, such as a pressure sensor (e.g., accumulator pressure sensor 107). Thus, as illustrated in FIG. 1B, the controller 400 receives sensor signals, such as a speed signal from the speed sensor 201 and accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an implementation, a passive cooling system may be used that takes advantage of the mechanical power produced by motor 220. Referring to FIGS. 1D and 8E, compression system 200 includes motor 220 having an external rotating armature 230. Specifically, armature 230 of motor 220 (e.g., a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature 230 causes the air transfer device 240 to create an air flow that passes over at least a portion of the motor. In an implementation, air transfer device 240 includes one or more fan blades coupled to the external armature 230. In an implementation, a plurality of fan blades may be arranged in an annular ring such that the air transfer device 240 acts as an impeller that is rotated by movement of the external rotating armature 230. As depicted in FIGS. 1D and 8E, air transfer device 240 may be mounted to an outer surface of the external armature 230, in alignment with the motor 220. The mounting of the air transfer device 240 to the armature 230 allows air flow to be directed toward the main portion of the external rotating armature 230, providing a cooling effect during use. In an implementation, the air transfer device 240 directs air flow such that a majority of the external rotating armature 230 is in the air flow path.

Further, referring to FIGS. 1D and 8E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device 240, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled exhaust gases 327 vented from canister system 300 are directed toward power supply 180 and toward compression system 200. In an implementation, base 315 of canister system 300 receives the exhaust gases from the canisters. The exhaust gases 327 are directed through base 315 toward outlet 325 of the base and toward power supply 180. The exhaust gases, as noted, are cooled due to decompression of the gases and therefore passively provide cooling to the power supply 180. When the compression system is operated, the air transfer device 240 will gather the cooled exhaust gas and direct the exhaust gas toward the motor of compression system 200. Fan 172 may also assist in directing the exhaust gas across compression system 200 and out of the housing 170. In this manner, additional cooling may be obtained without requiring any further power from the battery.

### Canister System

Oxygen concentrator 100 may include at least two canisters, each canister including a gas separation adsorbent. The canisters of oxygen concentrator 100 may be disposed formed from a molded housing. In an implementation, canister system 300 includes two housing components 310 and 510, as depicted in FIG. 1I. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent welded together.

As shown, valve seats 322, 324, 332, and 334 and air pathways 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, filter 129 may be placed into respective canister receiving portions of housing components 310 and 510 facing the inlet of the respective canisters. The filter 129 removes particles from the feed gas stream entering the canisters.

In some implementations, pressurized air from the compression system 200 may enter air inlet 306. Air inlet 306 is coupled to inlet conduit 330. Air enters housing component 310 through inlet 306 and travels through inlet conduit 330, and then to valve seats 322 and 324. FIG. 1J and FIG. 1K depict an end view of housing component 310. FIG. 1J depicts an end view of housing component 310 prior to fitting valves to housing component 310. FIG. 1K depicts an end view of housing component 310 with the valves fitted to the housing component 310. Valve seats 322 and 324 are configured to receive inlet valves 122 and 124 respectively. Inlet valve 122 is coupled to canister 302 and inlet valve 124 is coupled to canister 304. Housing component 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from inlet conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing component 310 into canister 302. Similarly valve seat 324 includes an opening 375 that passes through housing component 310 into canister 302. Air from inlet conduit 330 passes through openings 323 or 375 if the respective valves 122 and 124 are open, and enters a canister.

Check valves 142 and 144 (See FIG. 1I) are coupled to canisters 302 and 304, respectively. Check valves 142 and 144 are one way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing component 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing component 310.

Oxygen enriched air from either canister travels through conduit 342 or 344 and enters conduit 346 formed in housing component 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 1B, gas pressure within the accumulator 106 may be measured by a sensor, such as with an accumulator pressure sensor 107. (See also FIG. 1F.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor may alternatively measure pressure of the gas outside of the accumulator 106, such as in an output path between the accumulator 106 and a valve (e.g., supply valve 160) that controls the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to desorb nitrogen from the adsorbent. Canister 302 is vented by closing inlet valve 122 and opening outlet valve 132. Outlet valve 132 releases the exhaust gas from canister 302 into the volume defined by the end of housing component 310. Foam material may cover the end of housing component 310 to reduce the sound made by release of gases from the canisters. Similarly, canister 304 is vented by closing inlet valve 124 and opening outlet valve 134. Outlet valve 134 releases the exhaust gas from canister 304 into the volume defined by the end of housing component 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

In an exemplary implementation, a portion of the oxygen enriched air may be transferred from canister 302 to canister 304 when canister 304 is being vented of nitrogen. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. Flow of oxygen enriched air between the canisters is controlled using flow restrictors and valves, as depicted in FIG. 1B. Three conduits are formed in housing component 510 for use in transferring oxygen enriched air between canisters. As shown in FIG. 1L, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 1M. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 1M. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen purity (fractional oxygen concentration, typically expressed as a percentage) of the gas passing through the chamber.

### Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in an oxygen accumulator 106 prior to being provided to a user. In some implementations, a tube may be coupled to the accumulator 106 to provide the oxygen enriched air to the user. Oxygen enriched air may be provided to the user through an airway delivery device that transfers the oxygen enriched air to the user's mouth and/or nose. In an implementation, an outlet may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 1F. In an implementation, expansion chamber 162 may include one or more devices configured to estimate an oxygen purity of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.25 D flow restrictor. Other flow restrictor types and sizes may be used. In some implementations, the diameter of the air pathway in the housing may be restricted to create restricted gas flow. Flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste.

Expansion chamber 162 may include one or more oxygen sensors adapted to determine an oxygen purity of gas passing through the chamber. In an implementation, the oxygen purity of gas passing through expansion chamber 162 is estimated using an oxygen sensor 165. An oxygen sensor is a device configured to measure oxygen purity of a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic oxygen sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen purity may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method”, which published as U.S. Publication No. 2009/0065007 A1 on Mar. 12, 2009 and is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based based on the breathing rate and/or breathing volume of the user, as estimated by flow rate sensor 185.

In some implementations, ultrasonic oxygen sensor 165 and, for example, flow rate sensor 185 may provide a measurement of an actual amount of oxygen being provided. For example, flow rate sensor 185 may measure a volume of gas (based on flow rate) provided and ultrasonic oxygen sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc. prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through delivery conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as to detect the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

Oxygen enriched air may be provided to a user through delivery conduit 192. In an implementation, delivery conduit 192 may be a silicone tube. Delivery conduit 192 may be coupled to a user using an airway delivery device 196, as depicted in FIGS. 1G and 8H. An airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device (e.g., a nasal cannula). As depicted in FIG. 1H, oxygen enriched air may be provided to a user through both nasal cannula airway delivery device 196 and mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, mouthpiece 198 is used in combination with nasal cannula airway delivery device 196 to provide oxygen enriched air to a user, as depicted in FIG. 1H. Both mouthpiece 198 and nasal cannula airway delivery device 196 are coupled to an inhalation sensor. In one implementation, mouthpiece 198 and nasal cannula airway delivery device 196 are coupled to the same inhalation sensor. In an alternate implementation, mouthpiece 198 and nasal cannula airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both mouthpiece 198 and nasal cannula airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, as described below. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g. programmed by program instructions) to operate oxygen concentrator 100 and is further configured to monitor the oxygen concentrator 100 such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen purity of the oxygen enriched air passing through expansion chamber 162. A minimum oxygen purity threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

FIG. 1O illustrates one implementation of a connected POC therapy system 450 including the POC 100. Controller 400 of the POC 100 includes the transceiver 430 configured to allow the controller 400 to communicate, using a wireless communication protocol such as the Global System for Mobile Telephony (GSM) or other protocol (e.g., WiFi), with a remote computing device such as a cloud-based server 460 such as over a network 470. The network 470 may be a wide-area network such as the Internet, or a local-area network such as an Ethernet. The controller 400 may also include a short range wireless module in the transceiver 430 configured to enable the controller 400 to communicate, using a short range wireless communication protocol such as Bluetooth™, with a portable computing device 480 such as a smartphone. The portable computing device, e.g. smartphone, 480 may be associated with a user 1000 of the POC 100.

The server 460 may also be in wireless communication with the portable computing device 480 using a wireless communication protocol such as GSM. A processor of the smartphone 480 may execute a program 482 known as an “app” to control the interaction of the smartphone 480 with the user 1000, the POC 100, and/or the server 460. The server 460 may have access to a database 466 that stores operational data about the POC 100 and user 1000.

The server 460 includes an analysis engine 462 that may execute methods of operating and monitoring the POC 100. The server 460 may also be in communication via the network 470 with other devices such as a personal computing device 464 via a wired or wireless connection. A processor of the personal computing device 464 may execute a “client” program to control the interaction of the personal computing device 464 with the server 460. One example of a client program is a browser.

Further functions that may be implemented with or by the controller 400 are described in detail in other sections of this disclosure.

### Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button is activated to turn the system off, controller 400 may initiate a shutdown sequence to place the system in a shutdown state (e.g., a state in which both canisters are pressurized).

Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). In other implementations, the number of flow rate settings may be increased or decreased. After a flow rate setting is selected, oxygen concentrator 100 will then control operations to achieve production of the oxygen enriched air according to the selected flow rate setting. Optionally, the control panel may include one or more hybrid button(s) to activate any of the hybrid modes described herein. Optionally, the control panel may include a POD button to activate a POD mode. Thus, the device may be set to operate in a traditional bolus mode (POD) where the device merely releases bolus for patient inspiration such as in accordance with the set flow rate, a continuous flow mode (CFM) where the device provides gas flow with gas characteristics that generally remain constant for inspiration and expiration such as in accordance with the set flow rate, and/or a hybrid mode where the gas characteristics generally change for inspiration and expiration as discussed herein. In some cases, the controller may automatically change from a mode of higher power consumption to a mode of lower power consumption based on remaining battery life. For example, on detection of a low battery condition such as when the controller is configured with a voltage detection circuit (e.g., an undervoltage detector) to sample battery voltage, the controller may switch from a continuous mode to a hybrid mode or a POD mode. Similarly, on detection of a low or lower battery condition, the controller may switch from hybrid mode to a POD mode.

Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

The user may manually indicate active mode or sleep mode by pressing button 630 for active mode or button 635 for sleep mode.

### Triggering the POC

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device, such as the server 460, forming part of the connected POC therapy system 450, as described above. In this latter implementation, the processors 410 may be configured by program instructions stored in the memory 420 of the POC 100 to transmit to the external computing device the measurements and parameters necessary for the performance of those steps that are to be carried out at the external computing device.

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., five flow rate settings). The controller 400 may implement a POD (pulsed oxygen delivery) or demand mode of operation. Controller 400 may regulate the volume of the one or more released pulses or boluses to achieve delivery of the oxygen enriched air according to the selected flow rate setting. The flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1.1 LPM.

Oxygen enriched air produced by oxygen concentrator 100 is stored in an oxygen accumulator 106 and, in a POD mode of operation, released to the user as the user inhales. The amount of oxygen enriched air provided by oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the wastage of oxygen, controller 400 may be programmed to open the supply valve 160 to release a bolus of oxygen enriched air soon after the onset of a user's inhalation is detected. For example, the bolus of oxygen enriched air may be provided in the first few milliseconds of a user's inhalation. Releasing a bolus of oxygen enriched air to the user as the user inhales may prevent wastage of oxygen by not releasing oxygen, for example, when the user is exhaling.

In an implementation, a sensor such as a pressure sensor 194 may be used to detect the onset of inhalation by the user and thereby trigger the release of a bolus. For example, the onset of inhalation may be detected by using pressure sensor 194. In use, delivery conduit 192 for providing oxygen enriched air is coupled to the user's nose and/or mouth through the nasal airway delivery device 196 and/or mouthpiece 198. The pressure in delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of delivery conduit 192, due, in part, to the venturi action of the air being drawn across the end of delivery conduit 192. Controller 400 analyses the pressure signal from the pressure sensor 194 to detect a drop in pressure indicating the onset of inhalation.

A positive change or rise in the pressure in delivery conduit 192 indicates an exhalation by the user. Controller 400 may analyze the pressure signal from pressure sensor 194 to detect a rise in pressure indicating the onset of exhalation. In one implementation, when a positive pressure change is sensed, supply valve 160 is closed until the next onset of inhalation is detected. Alternatively, supply valve 160 may be closed after a predetermined interval known as the bolus duration.

By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

### Hybrid Mode Oxygen Delivery

Hybrid mode therapy is a breath-synchronised therapy in which there is a non-zero inter-bolus flow of gas to the patient as well as boluses delivered in synchrony with inhalation as in POD mode. In such a mode, the controller may control operations of the device to activate delivery of the bolus at such synchrony times and otherwise control or operate the device to deliver the non-zero inter-bolus flow of gas. Thus, the device may provide a generally continuous flow of therapy gas during each respiratory cycle (i.e., inspiration and expiration) but the characteristic(s) of the gas flow (e.g., purity and/or flow rate) may differ during inspiration (or part of inspiration) relative to non-inspiration times or expiration. Examples of such modes are described in more detail herein. Such gas characteristic delivery differences for the hybrid mode(s) may be implemented with multiple flow paths within the oxygen concentrator that employ different configurations. For example, such gas characteristic delivery differences may be implemented with a primary flow path (or primary path) and one or more secondary flow paths (or secondary paths). In this regard, the primary path generally concerns the typical path for flow of therapy gas from the accumulator through the supply valve that releases inspiratory triggered boluses to the delivery conduit. The primary path may provide the therapy gas to the delivery conduit with a first gas characteristic. Moreover, a secondary path generally concerns a path for flow of therapy gas to the delivery conduit that is separate from the primary path. Such a secondary path may provide the therapy gas to the delivery conduit with a second gas characteristic that is different from the first gas characteristic. In some example implementations, therapy gas provision via the primary path may generally involve therapy gas provided for inspiration times, whereas therapy gas provision via the secondary path(s) may generally involve therapy gas provided for expiration times or non-inspiratory times. However, in other examples, the secondary path(s) may also provide therapy gas for inspiratory times. Examples of such different paths for different hybrid modes are discussed in more detail herein.

### Bilevel Purity

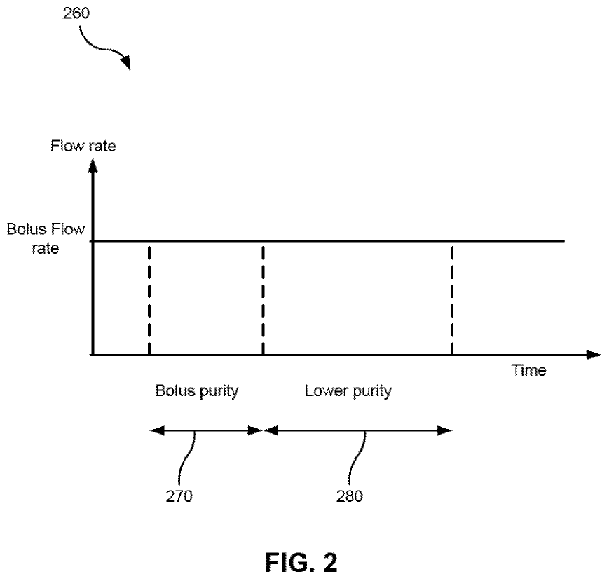

FIG. 2 contains a graph 260 illustrating an example of a hybrid mode, referred to as bilevel purity. In bilevel purity hybrid mode, each bolus of oxygen enriched air is released in synchrony with inhalation, as in POD mode, at a flow rate referred to as the bolus flow rate and at an oxygen purity referred to as the bolus purity. As such, the bolus purity may be equivalent to the oxygen purity of oxygen enriched air. This is illustrated by the period 270 in the graph 260, wherein the period 270 represents a period of time at which the device operates to produce gas flow so that the patient is provided a flow of gas at the bolus flow rate at the bolus purity. However, in between periods of bolus release referred to as inter-bolus periods, such as the period 280, the device operates to produce gas flow so that the patient is provided a flow of gas at the bolus flow rate, except at lower oxygen purity.

The lower oxygen purity of the inter-bolus flow means less oxygen is wasted than during conventional continuous flow, in which the oxygen purity and flow rate are generally constant. This in turn helps to extend battery life, since the device, including the compressor, does not need to work as hard as during conventional continuous flow to maintain system pressure at the desired value for the current flow rate setting. In addition, portable oxygen concentrators are limited in the volume of oxygen they can produce in a given time due to the design constraints (size, weight, power consumption, adsorbent mass). By conserving oxygen delivery, bilevel purity hybrid mode allows the other design constraints more room for optimisation.

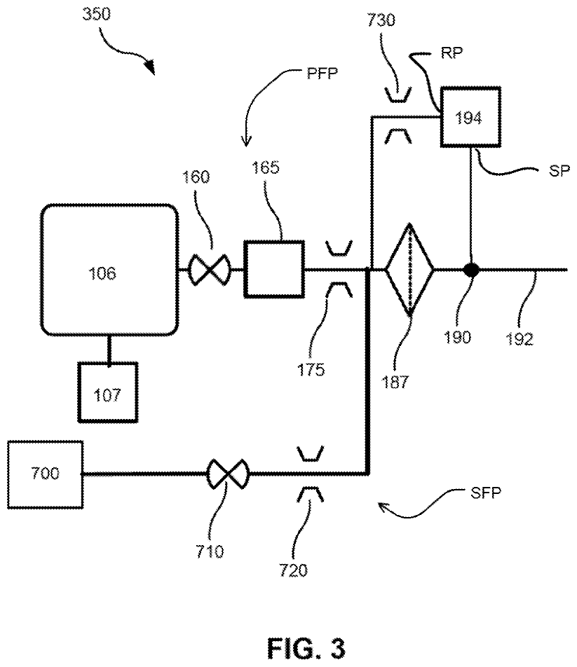

FIG. 3 is a schematic of a modification of the outlet system of FIG. 1F, according to one implementation of bilevel purity hybrid mode. The modified outlet system 350 of FIG. 3 is the same as that illustrated in FIG. 1F, except with new elements: a flow source 700, secondary valve 710, such as a two-way or two-port valve, a flow restrictor 720, and a restrictor 730. The flow rate sensor 185 may be omitted from the modified outlet system 350 as illustrated in FIG. 3, or optionally may be included after the flow restrictor 175 as illustrated in FIG. 1F.

The flow source 700 may be coupled to the downstream side of the flow restrictor 175 via a secondary flow path (SFP) comprising the secondary valve 710 and the flow restrictor 720. The secondary flow path is a different path from the primary flow path and may be operated to provide therapy gas with a different gas characteristic than the primary path. Thus, the flow in the secondary flow path is at a lower purity than the oxygen enriched air released by the supply valve 160 to the patient via the primary flow path (PFP). The controller 400 controls the secondary valve 710 to allow flow along the lower-purity path when a bolus is not being released by the supply valve 160. The controller 400 may also control the secondary valve 710 to prevent flow along the lower-purity path during bolus release. In other words, the secondary valve 710 may be actuated in anti-sync with the supply valve 160. As such, the controller 400 generates a control signal to control the secondary valve 710 to be open when the supply valve 160 is closed and closed when the supply valve 160 is open. In an alternative to the modified outlet system 350 for implementing bilevel purity hybrid mode, the two valves 160 and 710 may be replaced by a three-way valve (or a three-port valve) that is configured to couple the accumulator 106 to either the primary flow path (when triggered by the onset of inhalation) or the secondary, lower-purity path at all other times. The three-way valve may be either downstream of the flow restrictors 175 and 720 or upstream of a single flow restrictor which replaces and combines the effects of the flow restrictors 175 and 720.

In one implementation, the flow source 700 may be the compressor 210 with an outlet to the secondary path. In such an implementation, the flow restrictor 720 is chosen such that the flow rate in the lower-purity path is approximately equal to the bolus flow rate in the higher-purity primary flow path (or primary path). In some implementations, the flow restrictor 720 may be omitted altogether, depending on the pressure of the flow source 700 and the pneumatic impedance of the secondary flow path.

In an alternative implementation, the flow source 700 may be a secondary compressor with an outlet to the secondary path. Such a compressor may be configured to generate a flow of air at flow rates approximately equal to the bolus flow rates in the higher-purity path. In such an implementation the flow restrictor 720 may be omitted. The secondary compressor may optionally be controlled by the controller 400 to achieve the specified flow rates.

In either such implementation, the oxygen purity in the lower-purity path is approximately that of ambient air (21%).

In yet a further implementation of bilevel purity hybrid mode, the flow source 700 is a portion of the vented exhaust gas that has been re-routed from the outlet 130 (e.g., from the exhaust outlet of the canister(s)) to the lower-purity path. Such vented exhaust gas may be of oxygen purity typically around the ambient purity of 21%, but may be as high as 35% and as low as 4% depending on the amount of the purge flow. In one such implementation, the flow restrictor 720 is chosen such that the flow rate in the lower-purity path is approximately equal to the bolus flow rates in the higher-purity primary path. Thus, the therapy gas provided to the delivery conduit in such a hybrid mode may use both accumulated enriched gas (e.g., a bolus) and exhaust gas that may flow to the delivery conduit at least during patient inspiration and patient expiration. The hybrid mode may then vary a characteristic of the therapy gas, such as where the varied characteristic is oxygen purity. The varied oxygen purity may have a first oxygen purity during at least a portion of patient inspiration and a second oxygen purity after the portion of patient inspiration (e.g., during remaining inspiration and/or expiration). The first oxygen purity may be a purity in a range of about 50 percent to about 99 percent, which may be attributable to the bolus release gas and may be provided via the primary path to the delivery conduit. Moreover, the second oxygen purity may be a purity in a range of about 4 percent to 35 percent, which may be attributable to the vented exhaust gas and may be provided via the secondary path to the delivery conduit. Thus, the primary path, which generally concerns the path for flow of therapy gas from the accumulator via the supply valve that releases inspiratory triggered boluses to the delivery conduit, may provide the therapy gas with the first oxygen purity. Moreover, the secondary path, which is a flow path to the delivery conduit that is separate from the primary path, may provide the therapy gas with the second oxygen purity.

In some such implementations, the sensor configuration of the outlet system of FIG. 1F may be modified in the modified outlet system 350. For example, the pressure sensor 194 in the outlet system of FIG. 1F typically is differentially connected, so that it has its “sense port” (SP) connected to connector 190 or elsewhere in the delivery conduit 192 and it has its “reference port” (RP) connected to ambient (not shown in FIG. 1F). This sensor configuration may be modified in the modified outlet system 350 so that the reference port (RP) is instead within the system such as being coupled downstream of the supply valve. For example, it may be connected to the downstream side of a flow restrictor 730. The upstream side of the flow restrictor 730 is connected to the downstream side of the flow restrictor 175. With such a differential connection, the modified outlet system 350 may be able to trigger more accurately than if the pressure sensor 194 were connected as in FIG. 1F. The lower-purity flow through the secondary path in the inter-bolus periods causes the pressure at the connector 190, and therefore the sense port of the pressure sensor 194, to be elevated substantially above ambient just before the onset of inhalation. If the reference port of the pressure sensor 194 were otherwise connected to ambient, the substantially positive pressure difference between the ports of the pressure sensor 194 might saturate the pressure sensor 194 just before the onset of inhalation, making it more difficult to reliably sense the drop in pressure at the connector 190 resulting from the onset of inhalation.

However, with the differential connection of FIG. 3, the pressure difference between the ports of the pressure sensor 194 is much smaller, just before the onset of inhalation, and in fact may even be slightly negative. The pressure sensor 194 therefore remains unsaturated. Because of the flow restrictor 730, the dynamic or adaptive reference pressure is in a sense a damped or lagged version of the pressure at the connector 190. The onset of inhalation causes the pressure at the sense port (the connector 190) to drop sharply, while due to the flow restrictor 730 the pressure at the reference port stays constant for a short interval after the onset of inhalation. The pressure difference across the ports of the pressure sensor 194 is therefore pulled in the negative direction for long enough to be detected by the controller 400. The modified reference port connection effectively acts as a dynamic or adaptive threshold against which the pressure at the connector 190 is compared to detect the onset of inhalation.

Optionally, the device may be controlled so that the bilevel purity hybrid mode can be deactivated. Thus, with the aforementioned secondary valve configuration(s), oxygen enriched air is not required to be produced in bilevel purity hybrid at all times using the modified outlet system 350. In some implementations, the controller 400 may maintain the secondary valve 710 in a closed state so that the oxygen enriched air can be delivered according to a different mode without use of the secondary path. For example, with the maintained closure state, the controller can operate the device to provide gas flow in a POD mode via the primary path. Optionally, the controller may be configured to operate in the POD mode until a control (e.g., a hybrid button or a comfort button) on the control panel 600 is activated. For example, the control may be activated if the user is experiencing dyspnea or shortness of breath and is in need of reassurance or comfort. Once such a control is activated, the controller 400 may generate control signals to operate the secondary valve such as to begin to open and close the secondary valve 710 in anti-sync with the supply valve 160 as described above to implement bilevel purity hybrid mode. Optionally, pressing of the button may trigger operation in the hybrid mode for a predetermined period or for an indefinite period until the control on the control panel is de-activated. For example, the comfort button may activate the hybrid mode for such a predetermined time period. The controller 400 then reverts to control of oxygen enriched air in a POD mode after the predetermined time. Pressing the hybrid button may activate the hybrid mode in a more continuous fashion such as until the user activates another mode or the device is turned off.

### Bilevel Flow Rate

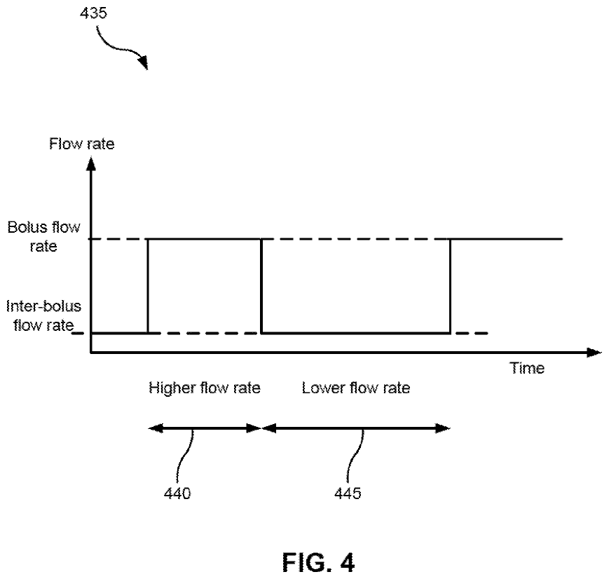

FIG. 4 contains a graph 435 illustrating another example of a hybrid mode, referred to as bilevel flow rate. In bilevel flow rate hybrid mode, each bolus of oxygen enriched air is released in synchrony with inhalation, as in POD mode and bilevel purity hybrid mode, at the bolus flow rate. This is illustrated by the period 440 in the graph 435. However, during inter-bolus periods such as the period 445, the device operates to produce gas flow so that the patient is provided with a flow of gas at the bolus oxygen purity, except at a lower flow rate referred to as the inter-bolus flow rate. As discussed in more detail herein, such a mode may be implemented with a primary flow path and a secondary flow path. To achieve the different gas flow rate characteristics, the paths may be configured with different flow characteristics.

The lower flow rate of the inter-bolus flow means less oxygen is wasted than during conventional continuous flow, in which the flow rate and oxygen purity are generally constant over the breathing cycle. This in turn helps to extend battery life, since the device, including the compressor, does not need to work as hard as during conventional continuous flow to maintain system pressure at the desired value for the current flow rate setting. In addition, portable oxygen concentrators are limited in the volume of oxygen they can produce in a given time due to the design constraints (size, weight, power consumption, adsorbent mass). By conserving oxygen delivery, bilevel flow rate hybrid mode allows the other design constraints more room for optimisation.

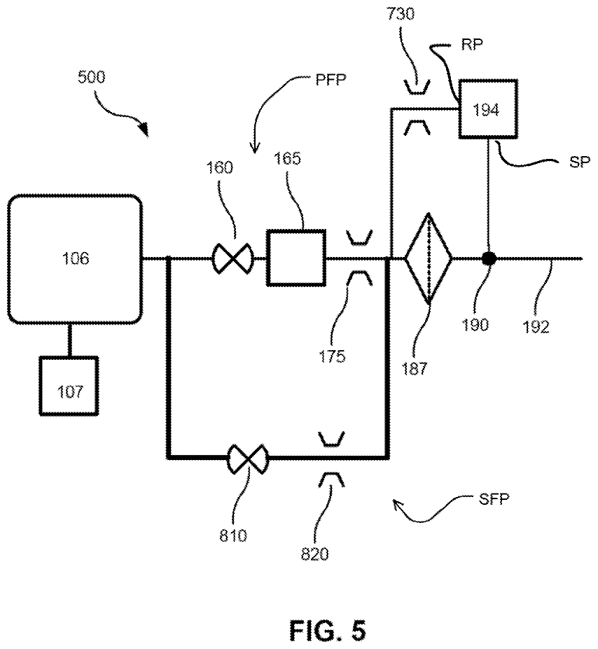

FIG. 5 is a schematic of a modification of the outlet system of FIG. 1F, according to one implementation of bilevel purity hybrid mode. The modified outlet system 500 of FIG. 5 is similar to the modified outlet system 350 illustrated in FIG. 3, except that instead of receiving flow from the flow source 700 like the secondary valve 710, a secondary valve 810 (e.g., a two-way or two port valve) receives flow from the accumulator 106. In other words, the secondary valve 810 and the flow restrictor 820, which may be placed in any order, form a secondary flow path (SFP) for the oxygen enriched air from the accumulator 106. The flow restrictor 820 is chosen so that the secondary flow path is a lower-flow path. That is, the flow of the secondary path is substantially lower than the bolus flow rate in the primary flow path (PFP).

The controller 400 is configured to control the secondary valve 810 to allow flow along the lower-flow path when the controller is not controlling a release of a bolus with the supply valve 160. The controller 400 may also control the secondary valve 810 to prevent flow along the lower-flow path during that controlled bolus release. In other words, the secondary valve 810 may be actuated in anti-sync with the supply valve 160. As such, the controller 400 generates a control signal to control the secondary valve 710 to be open when the supply valve 160 is closed and closed when the supply valve 160 is open.

The modified outlet system 500 may also implement the differentially connected pressure sensor 194 with the flow restrictor 730, as in the modified outlet system 350, to enable more accurate triggering.

Optionally, the device may be controlled so that the bilevel flow rate hybrid mode can be deactivated. Thus, with the aforementioned secondary valve configuration(s), oxygen enriched air is not required to be produced in bilevel flow rate hybrid mode at all times using the modified outlet system 500. In some implementations, the controller 400 may maintain the secondary valve 810 in a closed state so that the oxygen enriched air can be delivered according to a different mode without use of the secondary path. For example, with the maintained closure state, the controller can operate the device to provide gas flow in a POD mode via the primary flow path (or primary path). Similar to the operations previously described, the controller 400, such as in response to a user pressing a control button (e.g., a comfort button or a hybrid button), can operate in the bilevel flow rate hybrid mode by generating control signals to the aforementioned valves, either for a predetermined period of time or in a more continuous fashion as previously described.

In an alternative implementation, the modified outlet system 500 may be configured for providing the bilevel flow rate hybrid mode without the secondary valve 810. With the secondary valve 810 removed, the secondary, lower-flow path through the flow restrictor 820 provides a gas flow as long as the POC 100 itself is operating. To permit a lower flow rate of the secondary path relative to the primary path, the paths may be configured with different flow characteristics such that a flow characteristic of the primary path is different from a flow characteristic of the secondary path. For example, a flow restrictor of the secondary path may be chosen to restrict flow so as to achieve a lower flow rate of gas in the secondary path when compared to the flow rate of the primary path. Similarly, the pneumatic resistance of the primary and secondary flow paths may be chosen, such as according to different conduit sizes, to achieve the flow rate differences. For example, a smaller, more restrictive conduit may be chosen for the secondary path when compared to the conduit of the primary path.

In a further alternative modified outlet system for implementing bilevel flow rate hybrid mode, the valves 160 and 810 may be replaced by a three-way valve (e.g., three-port valve) that pneumatically couples the accumulator 106 to the primary path and the secondary flow path. Thus, the three-way valve may be activated by the controller to selectively pneumatically couple the accumulator to one of the primary path, such as when the controller is triggered by detection of the onset of inhalation, and the secondary, lower-flow path, such as at all other times.

One benefit of the bilevel flow rate hybrid delivery mode is that the oxygen enriched air delivered at a low flow rate via the secondary, lower-flow path “pools” within the delivery conduit 192 and is therefore available for inhalation as soon as inhalation begins, even before the opening of the primary path for the release of the bolus.

### Intermediate Implementations

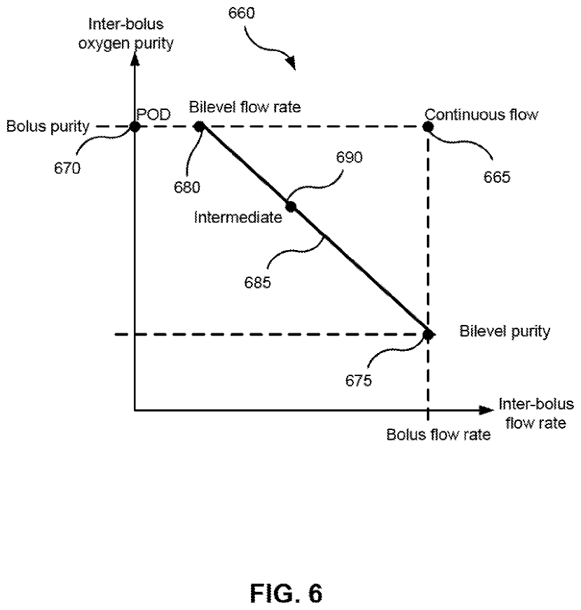

FIG. 6 contains a graph 660 illustrating various modes of delivery of oxygen enriched air by an oxygen concentrator. The horizontal axis represents the inter-bolus flow rate and the vertical axis represents the inter-bolus oxygen purity. The point 665 represents continuous flow delivery, in which the inter-bolus flow rate equals the bolus flow rate and the inter-bolus purity is the same as the purity of the oxygen enriched air, i.e. the bolus purity (e.g. 93%). The point 670 represents POD mode, in which the inter-bolus flow rate is zero. The point 675 represents the bilevel purity species of hybrid delivery mode, in which the inter-bolus flow rate is equal to the bolus flow rate but the inter-bolus purity is much reduced, typically to 21% for room air. The point 680 represents the bilevel flow rate species of hybrid delivery mode, in which the inter-bolus flow rate is substantially less than the bolus flow rate but the inter-bolus purity is the same as the bolus purity. The line 685 represents a progression of intermediate versions of a hybrid delivery mode between the bilevel purity species (point 675) and the bilevel flow rate species (point 680). The point 690 represents one such intermediate version in which the inter-bolus flow rate is somewhat less than the bolus flow rate and the inter-bolus purity is somewhat less than the bolus purity, while being greater than the purity of bilevel purity species.

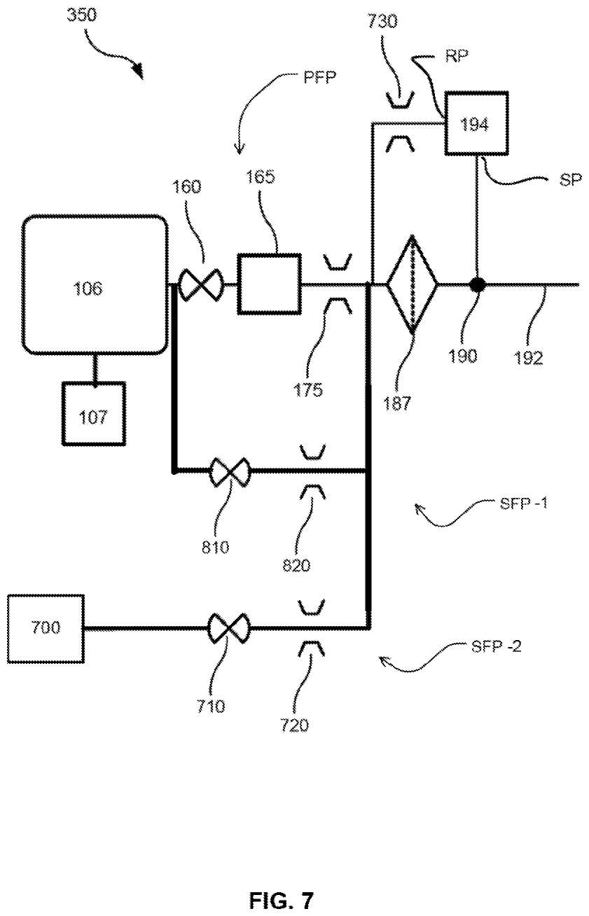

Such intermediate versions may be implemented with a controller 400 controlling a combination of components of the modified outlet systems 350 and 500 to implement both of the secondary lower-purity path (SPF from FIG. 3) and the secondary lower-flow path (SPF from FIG. 5). Such a combination of secondary paths (SFP-1, SFP-2) is illustrated in FIG. 7. In one such example, the controller 400 may generate control signals to control the secondary valves 710, 810, each of which are opened in anti-sync with the valve 160 of the primary path. The combination of the flows in the two secondary paths makes up the total inter-bolus flow. The respective sizes of the flow restrictors 720 and 820 may be chosen to set the flow rates in the two secondary paths and therefore the inter-bolus purity and flow rate to achieve the desired characteristics of the versions illustrated in the line of the graph FIG. 6.

Although the components of FIG. 7 illustrate the secondary valves 710 and 810, in some implementations one or both of these may be omitted.

The differentially connected pressure sensor 194 may be used with all examples of hybrid mode delivery in order to improve the accuracy of detection of inspiration and control of triggering of valve 160 for release of a bolus (and thereby the signals associated with the anti-sync operation of valves 710, 810.

### Glossary

For the purposes of the present technology disclosure, in certain forms of the present technology, one or more of the following definitions may apply. In other forms of the present technology, alternative definitions may apply.

### General

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen purity of 80% or greater.

Ambient: In certain forms of the present technology, the term ambient will be taken to mean (i) external of the treatment system or patient, and (ii) immediately surrounding the treatment system or patient.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m²=1 millibar˜0.001 atm). In this specification, unless otherwise stated, pressure is given in units of cmH₂O.

### General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

### LABEL LIST

| | |
|---|---|
| oxygen concentrator | 100 |
| inlet | 101 |
| air inlet | 105 |
| accumulator | 106 |
| pressure sensor | 107 |
| inlet muffler | 108 |
| inlet valve | 122 |
| inlet valve | 124 |
| filter | 129 |
| outlet | 130 |
| outlet valve | 132 |
| muffler | 133 |
| outlet valve | 134 |
| spring baffle | 139 |
| check valve | 142 |
| check valve | 144 |
| flow restrictor | 151 |
| valve | 152 |
| flow restrictor | 153 |
| valve | 154 |
| flow restrictor | 155 |
| supply valve | 160 |
| expansion chamber | 162 |
| oxygen sensor | 165 |
| ultrasonic emitter | 166 |
| ultrasonic receiver | 168 |
| outer housing | 170 |
| fan | 172 |
| outlet | 173 |
| outlet port | 174 |
| flow restrictor | 175 |
| power supply | 180 |
| flow rate sensor | 185 |
| particulate filter | 187 |
| connector | 190 |
| delivery conduit | 192 |
| pressure sensor | 194 |
| airway delivery device | 196 |
| mouthpiece | 198 |
| compression system | 200 |
| speed sensor | 201 |
| compressor | 210 |
| compressor outlet | 212 |
| motor | 220 |
| external rotating armature | 230 |
| air transfer device | 240 |
| compressor outlet conduit | 250 |
| graph | 260 |
| period | 270 |
| period | 280 |
| canister system | 300 |
| canister | 302 |
| canister | 304 |
| air inlet | 306 |
| housing component | 310 |
| base | 315 |
| valve seat | 322 |
| opening | 323 |
| valve seat | 324 |
| outlet | 325 |
| exhaust gases | 327 |
| inlet conduit | 330 |
| valve seat | 332 |
| valve seat | 334 |
| apertures | 337 |
| conduit | 342 |
| conduit | 344 |
| conduit | 346 |
| outlet system | 350 |
| opening | 375 |
| controller | 400 |
| processor | 410 |
| internal memory | 420 |
| transceiver | 430 |
| graph | 435 |
| period | 440 |
| period | 445 |
| POC therapy system | 450 |
| server | 460 |
| analysis engine | 462 |
| personal computing device | 464 |
| database | 466 |
| network | 470 |
| smartphone | 480 |
| program | 482 |
| outlet system | 500 |
| housing component | 510 |
| conduit | 530 |
| conduit | 532 |
| conduit | 534 |
| opening | 542 |
| opening | 544 |
| valve seat | 552 |
| valve seat | 554 |
| control panel | 600 |
| input port | 605 |
| power button | 610 |
| flow rate setting button | 620 |
| flow rate setting button | 622 |
| flow rate setting button | 624 |
| flow rate setting button | 626 |
| active mode button | 630 |
| mode button | 635 |
| altitude button | 640 |
| battery check button | 650 |
| relative battery power remaining LED | 655 |
| graph | 660 |
| point | 665 |
| point | 670 |
| point | 675 |
| point | 680 |
| line | 685 |
| point | 690 |
| flow source | 700 |
| secondary valve | 710 |
| flow restrictor | 720 |
| flow restrictor | 730 |
| secondary valve | 810 |
| flow restrictor | 820 |
| user | 1000 |

1. An oxygen concentrator for providing a therapy gas to a delivery conduit for patient inhalation, the oxygen concentrator comprising:
a compressor configured to generate a pressurised air stream;
one or more sieve beds, the one or more sieve beds comprising adsorbent material configured to preferentially adsorb a component gas from the pressurised air stream, thereby producing oxygen enriched air from the pressurised air stream;
a valve set configured to:
selectively pneumatically couple the compressor to the one or more sieve beds so as to selectively convey the pressurised air stream to the one or more sieve beds; and
selectively vent exhaust gas to atmosphere from an exhaust outlet of the one or more sieve beds;
an accumulator pneumatically coupled to the one or more sieve beds so as to receive the oxygen enriched air produced from a product outlet of the one or more sieve beds;
a supply valve configured to selectively release oxygen enriched air from the accumulator via a primary flow path and then to the delivery conduit;
a secondary flow path configured to pass a portion of the exhaust gas from the exhaust outlet to the delivery conduit; and
a controller operably coupled to the valve set and the supply valve, wherein the controller is configured to:
selectively actuate the valve set in a periodic pattern so as to produce oxygen enriched air for receiving by the accumulator and vent exhaust gas from the one or more sieve beds;
selectively actuate the supply valve to release oxygen enriched air from the accumulator to the delivery conduit in synchrony with inhalation of the patient,
wherein the therapy gas comprises the released oxygen enriched air and the portion of the exhaust gas.

2. The oxygen concentrator of claim 1 wherein the therapy gas is provided to the delivery conduit in a hybrid mode wherein the therapy gas flows to the delivery conduit at least during patient inspiration and patient expiration.

3. The oxygen concentrator of claim 2 wherein the hybrid mode varies a characteristic of the therapy gas.

4. The oxygen concentrator of claim 3 wherein the varied characteristic is oxygen purity.

5. The oxygen concentrator of claim 4 wherein the varied oxygen purity comprises a first oxygen purity during at least a portion of patient inspiration and a second oxygen purity after the portion of patient inspiration.

6. The oxygen concentrator of claim 5 wherein the first oxygen purity is a purity in a range of about 50 percent to about 99 percent, and the second oxygen purity is a purity in a range of about 4 percent to 35 percent.

7. The oxygen concentrator of any one of claims 5 to 6 wherein the primary flow path is configured to provide the therapy gas with the first oxygen purity and the secondary flow path is configured to provide the therapy gas with the second oxygen purity.

8. The oxygen concentrator of any one of claims 1 to 7 wherein the secondary flow path comprises a secondary valve configured to selectively release the portion of the exhaust gas to the delivery conduit, and wherein the controller is further configured to selectively actuate the secondary valve in anti-sync with actuation of the supply valve to release the portion of the exhaust gas to the delivery conduit.

9. The oxygen concentrator of claim 8, wherein the supply valve and the secondary valve are implemented as a three-way valve configured to release either the oxygen enriched air or the portion of the exhaust gas to the delivery conduit.

10. The oxygen concentrator of any of claims 1 to 9, further comprising a pressure sensor configured to generate a signal representative of a difference in pressure between a sense port and a reference port thereof, the sense port being connected to the delivery conduit, and the reference port being coupled to a flow path of the oxygen concentrator that is downstream of the supply valve.

11. The oxygen concentrator of claim 10, wherein the controller is further configured to detect onset of inhalation from the generated pressure difference signal and to actuate the supply valve based on the detected onset of inhalation.

12. The oxygen concentrator of claim 11, wherein the controller is configured to detect onset of inhalation by detecting a drop in the generated pressure difference signal.

13. The oxygen concentrator of claim 12, wherein the reference port of the pressure sensor is connected to a downstream side of the supply valve via a flow restrictor.

14. The oxygen concentrator of any one of claims 1 to 13, wherein the controller is configured to actuate the secondary valve in anti-sync with actuation of the supply valve in response to user activation of a control on an interface of the oxygen concentrator.

15. The oxygen concentrator of any one of claims 1 to 14, further comprising a flow restrictor within the secondary flow path and in line with the secondary valve.

16. The oxygen concentrator of claim 15, wherein the flow restrictor is configured such that a flow rate of exhaust gas when released to the delivery conduit is approximately equal to a flow rate of the oxygen enriched air when released to the delivery conduit.

17. The oxygen concentrator of any one of claims 1 to 16, when dependent on claim 8, further comprising a further secondary valve configured to selectively release oxygen enriched air from the accumulator to the delivery conduit via a flow restrictor, wherein the controller is further configured to selectively actuate the further secondary valve in anti-sync with actuation of the supply valve to release oxygen enriched air to the delivery conduit.

18. The oxygen concentrator of claim 17, when dependent on claim 2, wherein the hybrid mode varies a further characteristic of the therapy gas, wherein the varied further characteristic is flow rate of the therapy gas.

19. Apparatus for providing a therapy gas comprising:
means for generating a pressurised air stream;
means for preferentially adsorbing a component gas from the pressurised air stream, thereby producing oxygen enriched air from the pressurised air stream;
means for selectively pneumatically coupling, in a periodic pattern, the means for preferentially adsorbing with (a) the means for generating so as to selectively convey the pressurised air stream to the means for preferentially adsorbing, and (b) an exhaust outlet to atmosphere for selectively venting exhaust gas to atmosphere from the means for preferentially adsorbing, so as to produce oxygen enriched air within the means for preferentially adsorbing;
means for accumulating the oxygen enriched air produced from a product outlet of the means for preferentially adsorbing;
means for selectively releasing oxygen enriched air from the means for accumulating to a delivery conduit for a patient in synchrony with inhalation of the patient; and
means for passing a portion of the exhaust gas to the delivery conduit,
wherein the therapy gas comprises the released oxygen enriched air from the means for accumulating and the portion of the exhaust gas.

20. An oxygen concentrator for producing a therapy gas for a patient, the oxygen concentrator comprising:
a compressor configured to generate a pressurised air stream;
one or more sieve beds, the one or more sieve beds comprising adsorbent material configured to preferentially adsorb a component gas from the pressurised air stream, thereby producing oxygen enriched air from the pressurised air stream;
a valve set configured to selectively pneumatically couple the compressor to the one or more sieve beds so as to selectively convey the pressurised air stream to the one or more sieve beds;
an accumulator pneumatically coupled to the one or more sieve beds so as to receive the oxygen enriched air produced by one or more sieve beds;
a supply valve configured to selectively release oxygen enriched air from the accumulator, via a primary path, to a delivery conduit for the patient;
a secondary valve configured to selectively release oxygen enriched air from the accumulator, via a secondary path, to the delivery conduit for the patient;
a controller operably coupled to the valve, the supply valve, and the secondary valve, the controller configured to:
selectively actuate the valve set in a periodic pattern so as to produce oxygen enriched air in the accumulator;
selectively actuate the supply valve to release oxygen enriched air to the delivery conduit in synchrony with inhalation of the patient; and
selectively actuate the secondary valve in anti-sync with actuation of the supply valve to release oxygen enriched air to the delivery conduit.

21. The oxygen concentrator of claim 20 wherein the therapy gas is provided to the delivery conduit in a hybrid mode wherein the therapy gas flows to the delivery conduit at least during patient inspiration and patient expiration; and wherein the hybrid mode varies a characteristic of the therapy gas.

22. The oxygen concentrator of claim 21 wherein the varied characteristic is a flow rate of the therapy gas, wherein a flow characteristic of the primary path is different from a flow characteristic of the secondary path.

23. The oxygen concentrator of any one of claims 20 to 22, further comprising a flow restrictor within the secondary path and in line with the secondary valve.

24. The oxygen concentrator of claim 23, wherein the flow restrictor is configured such that a flow rate of oxygen enriched air when released to the delivery conduit via the secondary valve is substantially lower than a flow rate of the oxygen enriched air when released to the delivery conduit via the supply valve.

25. The oxygen concentrator of any one of claims 22 to 24, wherein the supply valve and the secondary valve are implemented as a three-way valve configured to release oxygen enriched air to the delivery conduit.

26. The oxygen concentrator of any of claims 22 to 25, further comprising a pressure sensor configured to generate a signal representative of a difference in pressure between a sense port and a reference port thereof, wherein the sense port is connected to the delivery conduit and the reference port is coupled to a flow path of the oxygen concentrator that is downstream of the supply valve.

27. The oxygen concentrator of claim 26, wherein the controller is further configured to detect onset of inhalation from the generated pressure difference signal and to actuate the supply valve based on the detected onset of inhalation.

28. The oxygen concentrator of claim 27, wherein the controller is configured to detect onset of inhalation by detecting a drop in the generated pressure difference signal.

29. The oxygen concentrator of claim 28, wherein the reference port of the pressure sensor is connected to a downstream side of the supply valve via a flow restrictor.

30. The oxygen concentrator of any one of claims 22 to 29, wherein the controller is configured to actuate the secondary valve in anti-sync with actuation of the supply valve in response to user activation of a control on an interface of the oxygen concentrator.

31. The oxygen concentrator of any one of claims 22 to 30, further comprising a further secondary valve configured to selectively release a portion of exhaust gas from the one or more sieve beds to the delivery conduit, wherein the controller is further configured to selectively actuate the further secondary valve in anti-sync with actuation of the supply valve to release the portion of the exhaust gas to the delivery conduit.

32. The oxygen concentrator of claim 31, when dependent on claim 21, wherein the hybrid mode varies a further characteristic of the therapy gas, wherein the varied further characteristic is oxygen purity of the therapy gas.

33. Apparatus comprising:
means for generating a pressurised air stream;
means for preferentially adsorbing a component gas from the pressurised air stream, thereby producing oxygen enriched air from the pressurised air stream;
means for selectively pneumatically coupling, in a periodic pattern, the means for preferentially adsorbing with the means for generating to selectively convey the pressurised air stream to the means for preferentially adsorbing so as to produce oxygen enriched air in the means for preferentially absorbing;
means for accumulating the oxygen enriched air produced by the means for preferentially adsorbing;
primary means for selectively releasing, in synchrony with inhalation of a patient, oxygen enriched air from the means for accumulating to a delivery conduit for the patient; and
secondary means for selectively releasing, in anti-sync with actuation of the primary means for selectively releasing, oxygen enriched air from the means for accumulating to the delivery conduit for the patient.

34. An oxygen concentrator comprising:
a compressor configured to generate a pressurised air stream;
one or more sieve beds, the one or more sieve beds comprising adsorbent material configured to preferentially adsorb a component gas from the pressurised air stream, thereby producing oxygen enriched air from the pressurised air stream;
a valve set configured to selectively pneumatically couple the compressor to the one or more sieve beds so as to selectively convey the pressurised air stream to the one or more sieve beds;
an accumulator pneumatically coupled to the one or more sieve beds so as to receive the oxygen enriched air produced by the one or more sieve beds;
a supply valve configured to selectively release oxygen enriched air from the accumulator to a delivery conduit for a patient;
a secondary path configured to convey a flow of gas to the delivery conduit for the patient;
a pressure sensor configured to generate a signal representative of a difference in pressure between a sense port and a reference port thereof, the sense port being connected to the delivery conduit and the reference port being coupled to a flow path of the oxygen concentrator that is downstream of the supply valve; and
a controller operably coupled to the valve set and the supply valve, the controller configured to:
selectively actuate the valve set in a periodic pattern so as to produce oxygen enriched air for the accumulator;
detect onset of inhalation of the patient from the generated pressure difference signal; and
selectively actuate the supply valve to release oxygen enriched air to the delivery conduit in synchrony with inhalation of the patient.

35. The oxygen concentrator of claim 34, wherein the controller is further configured to actuate the supply valve based on the detected onset of inhalation.

36. The oxygen concentrator of any one of claims 34 to 35, wherein the controller is configured to detect onset of inhalation by detecting a drop in the generated pressure difference signal.

37. The oxygen concentrator of claim 36, wherein the reference port of the pressure sensor is connected to a downstream side of the supply valve via a flow restrictor.

38. The oxygen concentrator of any one of claims 34 to 37, wherein the secondary path comprises a secondary valve configured to selectively release exhaust gas from the one or more sieve beds to the delivery conduit.

39. The oxygen concentrator of claim 38, wherein the secondary path further comprises a further secondary valve configured to selectively release oxygen enriched air from the accumulator to the delivery conduit via a flow restrictor.

40. The oxygen concentrator of any one of claims 34 to 37, wherein the secondary path comprises a secondary valve configured to selectively release oxygen enriched air from the accumulator to the delivery conduit via a flow restrictor.

41. Apparatus comprising:
means for generating a pressurised air stream;
means for preferentially adsorbing a component gas from the pressurised air stream, thereby producing oxygen enriched air from the pressurised air stream;
means for selectively pneumatically coupling, in a periodic pattern, the means for preferentially adsorbing with the means for generating so as to selectively convey the pressurised air stream to the means for preferentially adsorbing so as to produce oxygen enriched air in the means for preferentially absorbing;
means for accumulating the oxygen enriched air produced by the means for preferentially adsorbing;
means for selectively releasing oxygen enriched air from the means for accumulating to a delivery conduit for a patient;
secondary means for conveying a flow of gas to the delivery conduit for the patient;
means for generating a signal representative of a difference in pressure between a sense port and a reference port thereof, the sense port being connected to the delivery conduit; and
means for detecting onset of inhalation of the patient from the generated pressure difference signal and for selectively actuating the means for selectively releasing oxygen enriched air to release oxygen enriched air to the delivery conduit in synchrony with inhalation of the patient.
